(19) United States
(12) Patent Application Publication
(10) Pub. No.: US 2018/0256401 A1
(43) Pub. Date: Sep. 13, 2018

(54) SURGICAL DEVICE TO ASSIST IN REPAIR OF SPLIT EARLOBES

(71) Applicant: Michael Jude Lacqua, Staten Island, NY (US)

(72) Inventor: Michael Jude Lacqua, Staten Island, NY (US)

(21) Appl. No.: 15/830,750

(22) Filed: Dec. 4, 2017

Related U.S. Application Data
(60) Provisional application No. 62/468,719, filed on Mar. 8, 2017.

(51) Int. Cl. A61F 11/00

(52) U.S. Cl. CPC A61F 11/004; A61B 17/32

(57) ABSTRACT

An adjustable, earlobe repair instrument acts as a guide to the surgeon when excising and ensures symmetry. The device fits over and behind the split segment of the earlobe. It is then secured, and holds the tissue firmly, enabling the surgeon to make clear, precise, symmetrical cuts when removing the healed split or cleft segment prior to repair.

### CROSS-REFERENCE TO RELATED APPLICATION

This application claims the benefit of priority of U.S. provisional application No. 62/468,719, filed on Mar. 8, 2017, the contents of which are herein incorporated by reference.

### BACKGROUND OF THE INVENTION

The current invention relates to tools for earlobe repair, and more particularly, to a surgical tool for assisting in repair of a split earlobe.

The details of earlobe repair surgery vary by person, depending on the patient's aesthetic concerns and the state of his or her earlobe. In general, earlobe repair surgery is performed as an outpatient procedure, with the use of a local anesthetic. To repair a single tear in the earlobe, the intervening earlobe split is excised and sutured from the front and back of the ear to repair the healed tear.

To properly excise the earlobe split, the surgeon must make symmetrical, three dimensional (3D) cuts of the patient's earlobe when removing the healed, split/cleft segment prior to repair. Surgeons often struggle with making the symmetrical cut due to a lack of tools for assisting in this specific type of procedure.

There exists a need for an instrument that acts as a guide to the surgeon when excising and ensures symmetry.

### BRIEF DESCRIPTION OF THE DRAWINGS

FIG. 1 is one embodiment of a perspective view of the invention;

FIG. 2 shows one embodiment of a front view of the invention;

FIG. 3 shows one embodiment of a front perspective view of the invention;

FIG. 4 shows one embodiment of a rear perspective view of the invention;

FIG. 5 shows one embodiment of a rear view of the invention;

FIG. 6 shows one embodiment of a rear view of the invention; and

FIG. 7 shows one embodiment of a rear perspective view of the invention.

### SUMMARY

An instrument for assisting in an earlobe split repair procedure includes a first limb and a second limb, wherein the first limb and the second limb are connected at their proximal ends at a first pivot point, the pivot point allowing the first limb and second limb to pivot with respect to each other. The instrument also includes a third limb and a fourth limb, wherein the third limb and the fourth limb are connected at their proximal ends at a second pivot point, the second pivot point allowing the third limb and the fourth limb to pivot with respect to each other. Also included are a first threaded rod connecting a distal end of the first limb to a distal end of the third limb, a first head of the first threaded rob extending from the distal end of the third limb, a second threaded rod connecting a distal end of the third limb to the fourth limb, a second head of the second threaded rod extending from the distal end of the fourth limb. The instrument also includes an angle adjustment rail through which the first head and second head are positioned, the angle adjustment rail allowing lateral movement of the first head and second head across the angle adjustment rail, the lateral movement allowing angular adjustment of the first limb and the second limb and the third limb and fourth limb.

In one embodiment, the first threaded rod and second threaded rod are loosened by rotating the first head and the second head in a counter-clockwise direction. In one embodiment, the first threaded rod and second threaded rod are locked by rotating the first head and the second head in a clockwise direction.

In one embodiment, the third and fourth limb include numerical markers. In one embodiment, the first limb and the third limb move together along the angle adjustment rail. In one embodiment, the second limb and the third limb move together along the angle adjustment rail.

In one embodiment, the earlobe fits within a center gap between a front limb pair of the first limb and a second limb, and a back-limb pair of the third limb and fourth limb.

In one embodiment, the first limb with the third limb and the second limb with the fourth limb are opened to fit to an edge of a split section of the earlobe.

Also described is a method of repairing a split earlobe by providing the described instrument, opening the first limb with the third limb and the second limb with the fourth limb to fit to an edge of a split section of the earlobe, rotating the first head and the second head in a clockwise direction to lock the first threaded rod and second threaded rod in place, and removing the split section of the earlobe that is between the limbs.

### DETAILED DESCRIPTION OF THE INVENTION

The following detailed description is of the best currently contemplated modes of carrying out exemplary embodiments of the invention. The description is not to be taken in a limiting sense, but is made merely for illustrating the general principles of the invention, since the scope of the invention is best defined by the appended claims.

As previously stated, surgeons often struggle with making symmetrical, 3D cuts of the patient's earlobe when removing the healed, split segment prior to repair due to a lack of assisting instrumentation. The invention claimed here solves this problem.

Broadly, one embodiment of the present invention is an instrument that acts as a guide to the surgeon when excising and ensures symmetry. The claimed device fits over and behind the split segment of the earlobe. It is then secured, and holds the tissue firmly, enabling the surgeon to make clear, precise, symmetrical cuts when removing the healed split segment prior to repair.

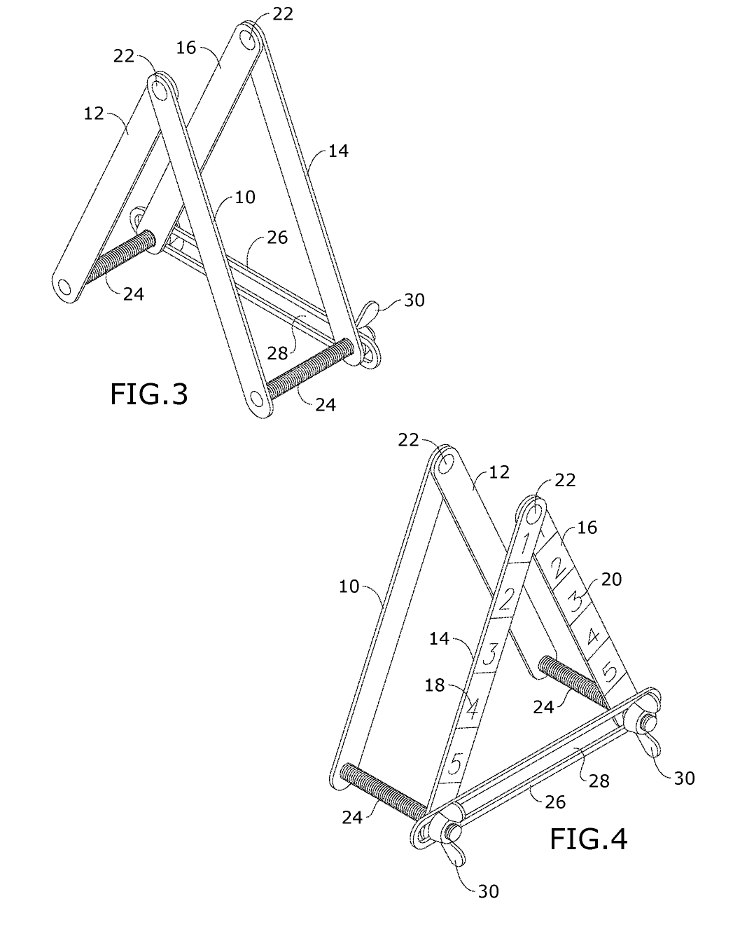

One embodiment of the claimed device is shown in FIG. 3. The device includes a first limb 10 and a second limb 12. The first limb 10 and second limb 12 are respectively connected to a third limb 14 and a fourth limb 16 with threaded rods 24. A proximal end of each limb includes pivot points 22. The pivot points 22 are an attachment point which allows the third limb 14 and fourth limb 16 to pivot with respect to each other and the first limb 10 and second limb 12 to pivot with respect to each other. In one embodiment, each limb can pivot from a closed position of zero degrees to 45 degrees to envelop the split earlobe.

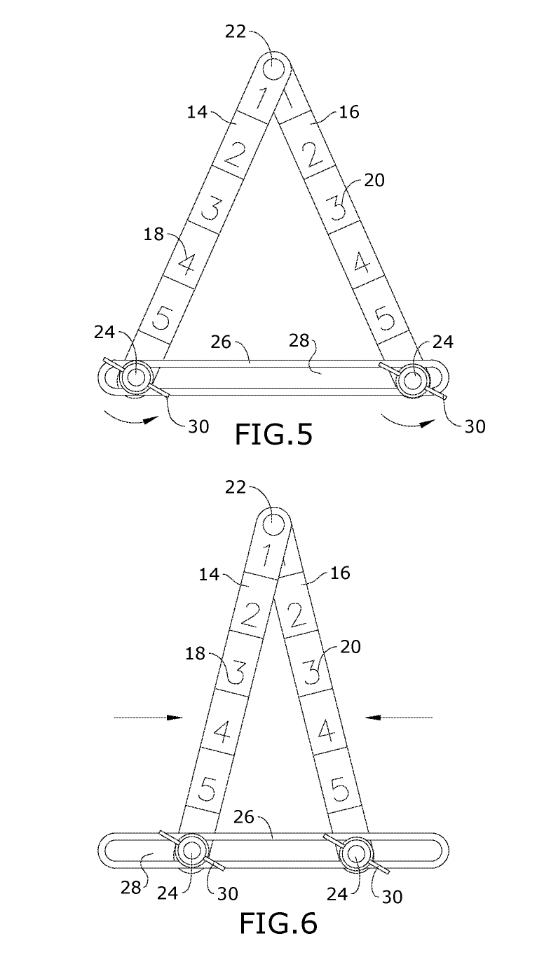

Connecting distal ends of third limb 14 and fourth limb 16 is an angle adjustment rod 26 with an angle adjustment rail 28. Threaded rod heads or wing mats 30 at an end of threaded rods 24 hold the threaded rods 24 in place through angle adjustment rail 28. Wing mats 30 can be tightened and loosened allowing angle adjustment of the first limb 10 and second limb 12 and third limb 14 and fourth limb 16 by allowing sliding of the wing mats 30 through angle adjustment rail 28. As shown in the rear perspective view of FIG. 4, third limb 14 includes third limb markers 18 and fourth limb 16 includes fourth limb markers 20. The markers 20 assist with assuring the third limb 14 and fourth limb 16 are symmetrically placed around the earlobe split or cleft.

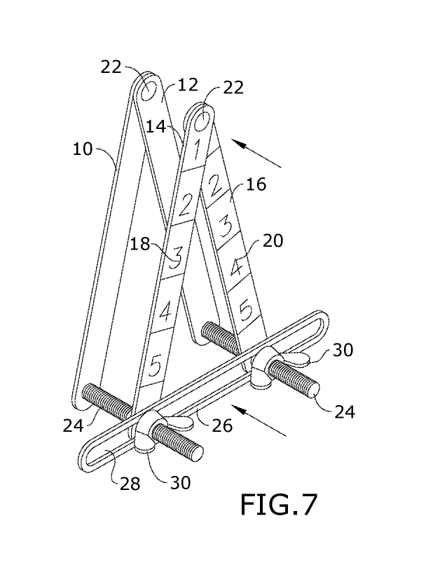

The embodiment of FIG. 5 shows the loosening of wing mats 30 by rotating them in a counter-clockwise direction. The angle is then adjusted and the limbs along adjustment rail 28 as shown in FIG. 6. The distance between the limbs is narrowed and the nuts 30 are then rotated clockwise and locked in place. The narrowed distance between limbs is shown in the rearview perspective of FIG. 7.

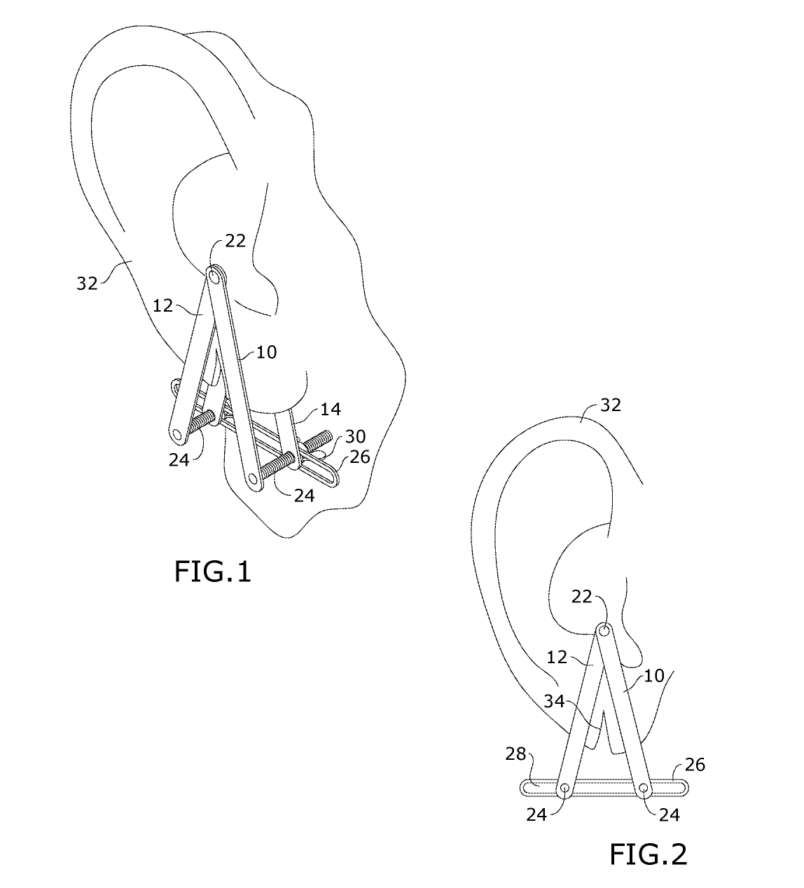

The device is shown in use on ear 32 in FIGS. 1 and 2. The limbs are opened to fit just to the outside edge of the split segment 34. The first limb 10 and third limb 14 move together along the angle adjustment rail 28, and the second limb 12 and fourth limb 16 move together along the angle adjustment rail 28. One in an appropriate place, the limbs are tightened and held by the wing mats 30 as previously described. Surgical cuts are made on the inside of the limbs to remove the healed, split segment evenly and symmetrically.

Advantageously, the device is one unit. It is opened and closed in the side to side and back to front direction to fit over any sized earlobe or defect. Once placed by the surgeon, it is tightened and secured, allowing the cuts to be made carefully, evenly and symmetrically.

In one embodiment, individual components are made in molds and then assembled by hand. In one embodiment, the components are made of plastic and are disposable. In another embodiment, the components are made of metal and resterilizable.

It should be understood, of course, that the foregoing relates to exemplary embodiments of the invention and that modifications may be made without departing from the spirit and scope of the invention as set forth in the following claims.

1. An instrument for assisting in an earlobe split repair procedure comprising:
a first limb and a second limb, wherein the first limb and the second limb are connected at their proximal ends at a first pivot point, the pivot point allowing the first limb and second limb to pivot with respect to each other;
a third limb and a fourth limb, wherein the third limb and the fourth limb are connected at their proximal ends at a second pivot point, the second pivot point allowing the third limb and the fourth limb to pivot with respect to each other;
a first threaded rod connecting a distal end of the first limb to a distal end of the third limb, a first head of the first threaded rob extending from the distal end of the third limb;
a second threaded rod connecting a distal end of the third limb to the fourth limb, a second head of the second threaded rod extending from the distal end of the fourth limb; and
an angle adjustment rail through which the first head and second head are positioned, the angle adjustment rail allowing lateral movement of the first head and second head across the angle adjustment rail, the lateral movement allowing angular adjustment of the first limb and the second limb and the third limb and fourth limb.

2. The instrument of claim 1, wherein the first threaded rod and second threaded rod are loosened by rotating the first head and the second head in a counter-clockwise direction.

3. The instrument of claim 1, wherein the first threaded rod and second threaded rod are locked by rotating the first head and the second head in a clockwise direction.

4. The instrument of claim 1, wherein the third and fourth limb include numerical markers.

5. The instrument of claim 1, wherein the first limb and the third limb move together along the angle adjustment rail.

6. The instrument of claim 1, wherein the second limb and the third limb move together along the angle adjustment rail.

7. The instrument of claim 1, wherein the earlobe fits within a center gap between a front limb pair of the first limb and a second limb, and a back-limb pair of the third limb and fourth limb.

8. The instrument of claim 1, wherein the first limb with the third limb and the second limb with the fourth limb are opened to fit to an edge of a split section of the earlobe.

9. A method of repairing a split earlobe comprising:
providing an alignment instrument, the instrument comprising;
a first limb and a second limb, wherein the first limb and the second limb are connected at their proximal ends at a first pivot point, the pivot point allowing the first limb and second limb to pivot with respect to each other;
a third limb and a fourth limb, wherein the third limb and the fourth limb are connected at their proximal ends at a second pivot point, the second pivot point allowing the third limb and the fourth limb to pivot with respect to each other;
a first threaded rod connecting a distal end of the first limb to a distal end of the third limb, a first head of the first threaded rob extending from the distal end of the third limb;
a second threaded rod connecting a distal end of the third limb to the fourth limb, a second head of the second threaded rod extending from the distal end of the fourth limb; and
an angle adjustment rail through which the first head and second head are positioned, the angle adjustment rail allowing lateral movement of the first head and second head across the angle adjustment rail, the lateral movement allowing angular adjustment of the first limb and the second limb and the third limb and fourth limb;
opening the first limb with the third limb and the second limb with the fourth limb to fit to an edge of a split segment of the earlobe;
rotating the first head and the second head in a clockwise direction to lock the first threaded rod and second threaded rod in place;
removing the split segment of the earlobe that is between the limbs.
